(19) United States
(12) Patent Application Publication
(10) Pub. No.: US 2026/0102542 A1
(43) Pub. Date: Apr. 16, 2026

(54) OSTOMY FILM

(71) Applicant: ConvaTec Limited, Flintshire (GB)

(72) Inventor: Leonardo Malcovati, Copenhagen (DK)

(21) Appl. No.: 19/419,371

(22) Filed: Dec. 15, 2025

Related U.S. Application Data
(63) Continuation of application No. PCT/US2024/033386, filed on Jun. 11, 2024.
(60) Provisional application No. 63/507,878, filed on Jun. 13, 2023.

(30) Foreign Application Priority Data
Aug. 10, 2023 (GB) 2312236.9

(51) Int. Cl. A61L 28/00; A61F 5/445

(52) U.S. Cl. CPC A61L 28/0011; A61F 5/445; A61L 28/0026

(57) ABSTRACT

An ostomy pouch that has a cavity for containing human waste defined by a multilayer barrier film that has, in order from adjacent to the cavity outwards, an inner polymer carrier layer, an inner tie layer, a barrier layer comprising poly(ethylene-co-vinyl alcohol), an outer tie layer, and an outer polymer carrier layer. The tie layers each have recycled polymer material and at least one compatibilizer.

### TECHNICAL FIELD OF THE INVENTION

The present invention relates to multilayer barrier films for ostomy applications.

### BACKGROUND TO THE INVENTION

An ostomy is a surgical procedure which is performed when a patient has lost normal bowel or bladder function as a result of a disease, injury or other defect. Most ostomy procedures are performed on cancer patients. After the ostomy procedure has been performed, waste from the patient is expelled via a surgical opening called a “stoma” into an ostomy appliance such as an ostomy pouch.

Since their invention in 1953, ostomy pouches have been made of several types of polymers; the main purpose of these polymers being to stop the odour of the contents of the pouch from diffusing into the environment.

In recent years, virtually all ostomy pouches on the market are made of coextruded multilayer films, in which one of the layers (referred to as the barrier layer) has low permeation to the odour components, whilst the other layers offer other functionalities, such as structural endurance, weldability, tactile and aesthetic feel, or allowing for compatibility of polymers with each other.

By far the most common polymer used as a barrier layer is poly (vinyl dichloride) (pVDC), which displays excellent barrier properties, but also presents disadvantages in that it can neither be processed by standard equipment, nor can it be blended with other commonly used polymers.

This makes trims, and other production scraps from pVDC-containing films impossible to recycle. Disposing of pVDC-containing products is also problematic, as its halogen content makes it ill-suited for incineration, which requires special scrubbers and additional energy to break the chlorine-carbon bonds. Due to its extreme chemical durability at room temperature, disposing of pVDC is environmentally irresponsible.

Other barrier materials have been tried, including polyamides, polynorbornenes, polypropylene; however, these have seen limited success.

There exists a need for new and improved multilayer films for ostomy applications which provide excellent odour barrier properties, and which can also be fully recycled.

It is an aim of embodiments of the present invention to overcome or mitigate at least one problem of the prior art, whether expressly described herein or not.

### SUMMARY OF THE INVENTION

According to a first aspect of the invention, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer.

Such a multilayer barrier film greatly limits the permeation ability of the main faecal odorant, hydrogen sulphide, and so allows for an ostomy pouch with excellent odour barrier properties. In addition, the film is fully recyclable and so allows for a highly environmentally friendly pouch. This recyclability further allows for scrap film material generated in the construction of new films to be reused in the manufacture of other films, thus leading to significant cost and material savings in the manufacture of such ostomy pouches. Further advantageously, the multilayer barrier film provides the ostomy pouch with excellent optical and mechanical properties.

At least one polymer carrier layer may comprise at least one chemically inert polymer. In some embodiments, the chemically inert polymer may be present in a total amount of at least 80 wt. % of the polymer carrier layer, or at least 85, or at least 90 wt. % of the polymer carrier layer. The polymer carrier layer may comprise the chemically inert polymer in a total amount of between 80-100 wt. % of the polymer carrier layer, or between 85-100, 90-100, or between 90-95 wt. % of the polymer carrier layer. In some embodiments, the chemically inert polymer may be present in an amount of 100% of the polymer carrier layer.

In some embodiments, at least one polymer carrier layer comprises at least one polymer that is independently chosen from: a polyolefin, a polyamide, and combinations thereof. In some embodiments, at least one polymer carrier layer comprises at least one polymer that is independently selected from the group consisting of: a polyolefin, a polyamide, and combinations thereof. In some embodiments, at least one polymer carrier layer may comprise a polyolefin. At least one polymer carrier layer may comprise at least one polymer that is independently chosen from: polyethylene, an ethylene copolymer, polypropylene, a propylene copolymer, and combinations thereof. At least one polymer carrier layer may comprise at least one polymer that is independently selected from the group consisting of: polyethylene, an ethylene copolymer, polypropylene, a propylene copolymer, and combinations thereof. The polymer carrier layer may comprise the polymer in a total amount as described for the chemically inert polymer above.

In some embodiments, at least one polymer carrier layer comprises polyethylene. At least one polymer carrier layer may comprise metallocene-catalysed polyethylene. At least one polymer carrier layer may comprise linear low-density polyethylene. At least one polymer carrier layer may preferably comprise metallocene-catalysed linear low-density polyethylene. The polymer carrier layer may comprise the polyethylene in a total amount as described for the chemically inert polymer above.

At least one polymer carrier layer may comprise at least one copolymer of ethylene with at least one of: hexene, octene, vinyl acetate, and combinations thereof. At least one polymer carrier layer may comprise ethylene-vinyl acetate (EVA). The EVA may have a vinyl acetate content of between 10-20% of the copolymer, or between 15-20% of the copolymer, or around 18% of the copolymer. The polymer carrier layer may comprise the ethylene copolymer in a total amount as described for the chemically inert polymer above.

The outer polymer carrier layer may preferably comprise at least one ethylene copolymer. The outer polymer carrier layer may comprise at least one copolymer of ethylene with at least one of: hexene, octene, vinyl acetate, and combinations thereof. The outer polymer carrier layer may preferably comprise ethylene-vinyl acetate (EVA). The EVA may have a vinyl acetate content of between 10-20% of the copolymer, or between 15-20% of the copolymer, or around 18% of the copolymer. The outer polymer carrier layer may comprise the ethylene copolymer in a total amount as described for the chemically inert polymer above.

The inner polymer carrier layer may preferably comprise polyethylene. The inner polymer carrier layer may comprise linear low-density polyethylene. The inner polymer carrier layer may preferably comprise metallocene-catalysed polyethylene, and more preferably metallocene-catalysed linear low-density polyethylene. The inner polymer carrier layer may comprise the polyethylene in a total amount as described for the chemically inert polymer above.

In preferred embodiments, the outer polymer carrier layer comprises at least one ethylene copolymer; and the inner polymer carrier layer comprises polyethylene, preferably metallocene-catalysed polyethylene, more preferably metallocene-catalysed linear low-density polyethylene. The outer polymer carrier layer may comprise at least one copolymer of ethylene with at least one of: hexene, octene, vinyl acetate, and combinations thereof; and the inner polymer carrier layer may comprise polyethylene, preferably metallocene-catalysed polyethylene, more preferably metallocene-catalysed linear low-density polyethylene. The outer polymer carrier layer may preferably comprise ethylene-vinyl acetate (EVA), and the inner polymer carrier layer may comprise polyethylene, preferably metallocene-catalysed polyethylene, more preferably metallocene-catalysed linear low-density polyethylene.

The inner polymer carrier layer may comprise a polymer material that is more impermeable to moisture than the polymer material of the outer carrier layer. This setup advantageously restricts the passage of moisture from the human waste contained in the cavity of the pouch through the inner polymer carrier layer, and so allows for the barrier layer to be kept largely free from moisture, whilst also allowing for moisture permeability through the outer carrier layer.

In some embodiments, at least one polymer carrier layer further comprises at least one pigment. The pigment may be present in an amount of between 0.1-10 wt. % of the carrier layer, or between 0.5-10 wt. %, or between 1-10 wt. %, or between 4-10 wt. % of the carrier layer.

In some embodiments, at least one polymer carrier layer has a thickness of at least 0.5 microns, or at least 1 micron, or at least 1.5, 2, 2.5, 3, 3.5, 4, 4.5, or at least 5 microns. In some embodiments, at least one polymer carrier layer has a thickness of no greater than 25 microns, or no greater than 24, 23, 22, 21, 20, 19, 18, 17, 16, or no greater than 15 microns. At least one polymer carrier layer may have a thickness of between 2-20 microns, or preferably between 4-18 microns, or between 6-16, or between 7-15 microns.

In some embodiments, the outer polymer carrier layer has a thickness of at least 4 microns, or at least 5, 6, 7, 8, 9, 10, 11, 12, or at least 13 microns. The outer polymer carrier layer may have a thickness of no greater than 25 microns, or no greater than 24, 23, 22, 21, 20, 19, 18, 17, 16, or no greater than 15 microns. The outer polymer carrier layer may have a thickness of between 8-20 microns, or preferably of between 10-18 microns, or between 12-16 microns, or between 13-15 microns.

In some embodiments, the inner polymer carrier layer has a thickness of at least 1 micron, or at least 2, 3, 4, 5, or at least 6 microns. The inner polymer carrier layer may have a thickness of no greater than 15 microns, or no greater than 14, 13, 12, 11, 10, 9, or no greater than 8 microns. In some embodiments, the inner polymer carrier layer has a thickness of between 2-12 microns, or preferably between 4-10 microns, or between 6-8 microns.

In some embodiments, the outer polymer carrier layer has a thickness of between 8-20 microns; and the inner polymer carrier layer has a thickness of between 2-12 microns, or between 4-10 microns, or between 6-8 microns. The outer polymer carrier layer may have a thickness of between 10-18 microns; and the inner polymer carrier layer may have a thickness of between 2-12 microns, or between 4-10 microns, or between 6-8 microns. The outer polymer carrier layer may have a thickness of between 12-16 microns; and the inner polymer carrier layer may have a thickness of between 2-12 microns, or between 4-10 microns, or between 6-8 microns. The outer polymer carrier layer may have a thickness of between 13-15 microns; and the inner polymer carrier layer may have a thickness of between 2-12 microns, or between 4-10 microns, or between 6-8 microns.

In some embodiments, the multilayer film comprises only 2 polymer carrier layers. In some embodiments, the multilayer film comprises 3 or at least 3 polymer carrier layers, or 4 or at least 4 polymer carrier layers, or 5 or at least 5 polymer carrier layers, or 6 or at least 6 polymer carrier layers, or 7 or at least 7 polymer carrier layers, or 8 or at least 8 polymer carrier layers, or 9 or at least 9 polymer carrier layers, or 10 or at least 10 polymer carrier layers.

The term “inner polymer carrier layer” refers to a polymer carrier layer of the multilayer film that is positioned nearer to the cavity of the ostomy pouch than the EVOH barrier layer.

In some embodiments, the multilayer film comprises a single inner polymer carrier layer. In other embodiments, the multilayer film comprises a plurality of inner polymer carrier layers. The film may comprise 2 or at least 2 inner polymer carrier layers, or 3 or at least 3 inner polymer carrier layers, or 4 or at least 4 inner polymer carrier layers, or 5 or at least 5 inner polymer carrier layers.

The multilayer film may comprise a first and second inner polymer carrier layer, the second inner polymer carrier layer being positioned nearer to the cavity of the ostomy pouch than the first inner polymer carrier layer. The first and second inner polymer carrier layers may be directly adjacent to each other. Alternatively, the first and second inner polymer carrier layers may not be directly adjacent to each other and may be separated by one or more layers of the multilayer film. In some embodiments, the first and second inner polymer carrier layers may be separated by a tie layer, said tie layer preferably comprising recycled polymer material and at least one compatibilizer.

In some embodiments, the multilayer film may comprise further inner polymer carrier layers, such as third, fourth, or fifth inner polymer carrier layers, with each consecutively numbered inner polymer carrier layer being positioned nearer to the cavity of the pouch than the previously numbered inner polymer carrier layer. In some embodiments, at least one inner polymer carrier layer is directly adjacent to at least one other inner polymer carrier layer. In some embodiments, all inner polymer carrier layers are directly adjacent to at least one other inner polymer carrier layer. In some embodiments, at least one inner polymer carrier layer is not directly adjacent to another inner polymer carrier layer, and said inner polymer carrier layer may be separated from at least one other inner polymer carrier layer by one or more layers of the multilayer film. Said inner polymer carrier layer may be separated from at least one other inner polymer carrier layer by a tie layer, said tie layer preferably comprising recycled polymer material and at least one compatibilizer. In some embodiments, all inner polymer carrier layers are not directly adjacent to another inner polymer carrier layer, and each inner polymer carrier layer may be separated from at least one other inner polymer carrier layer by one or more layers of the multilayer film. Each inner polymer carrier layer may be separated from at least one other inner polymer carrier layer by a tie layer, said tie layer preferably comprising recycled polymer material and at least one compatibilizer.

The term “outer polymer carrier layer” refers to a polymer carrier layer of the multilayer film that is positioned further away from the cavity of the ostomy pouch than the EVOH barrier layer.

In some embodiments, the multilayer film comprises a single outer polymer carrier layer. In other embodiments, the multilayer film comprises a plurality of outer polymer carrier layers. The film may comprise 2 or at least 2 outer polymer carrier layers, or 3 or at least 3 outer polymer carrier layers, or 4 or at least 4 outer polymer carrier layers, or 5 or at least 5 outer polymer carrier layers.

The multilayer film may comprise a first and second outer polymer carrier layer, the second outer polymer carrier layer being positioned further away from the cavity of the ostomy pouch than the first outer polymer carrier layer. The first and second outer polymer carrier layers may be directly adjacent to each other. Alternatively, the first and second outer polymer carrier layers may not be directly adjacent to each other and may be separated by one or more layers of the multilayer film. In some embodiments, the first and second outer polymer carrier layers may be separated by a tie layer, said tie layer preferably comprising recycled polymer material and at least one compatibilizer.

In some embodiments, the multilayer film may comprise further outer polymer carrier layers, such as third, fourth, or fifth outer polymer carrier layers, with each consecutively numbered outer polymer carrier layer being positioned further away from the cavity of the pouch than the previously numbered outer polymer carrier layer. In some embodiments, at least one outer polymer carrier layer is directly adjacent to at least one other outer polymer carrier layer. In some embodiments, all outer polymer carrier layers are directly adjacent to at least one other outer polymer carrier layer. In some embodiments, at least one outer polymer carrier layer is not directly adjacent to another outer polymer carrier layer, and said outer polymer carrier layer may be separated from at least one other outer polymer carrier layer by one or more layers of the multilayer film. Said outer polymer carrier layer may be separated from at least one other outer polymer carrier layer by a tie layer, said tie layer preferably comprising recycled polymer material and at least one compatibilizer. In some embodiments, all outer polymer carrier layers are not directly adjacent to another outer polymer carrier layer, and each outer polymer carrier layer may be separated from at least one other outer polymer carrier layer by one or more layers of the multilayer film. Each outer polymer carrier layer may be separated from at least one other outer polymer carrier layer by a tie layer, said tie layer preferably comprising recycled polymer material and at least one compatibilizer.

In some embodiments, the multilayer film comprises a greater number of outer polymer carrier layers than inner polymer carrier layers. In some embodiments, the multilayer film comprises a greater number of inner polymer carrier layers than outer polymer carrier layers. In some preferred embodiments, the multilayer film comprises the same number of outer polymer carrier layers as inner polymer carrier layers.

In some embodiments, the EVOH comprises between 10-50% vinyl alcohol units, or between 15-45%, or between 20-40%, or between 25-35%, or between 30-35% vinyl alcohol units.

In some embodiments, the barrier layer has a thickness of at least 1 micron, or at least 2, 3, 4, 5, 6, or at least 7 microns. In some embodiments, the barrier layer has a thickness of no greater than 25 microns, or no greater than 20, 15, or no greater than 10 microns. In some embodiments, the barrier layer has a thickness of between 3-15 microns, or between 3-12 microns, or between 4-11, 5-10, 6-9, or between 7-9 microns.

In some embodiments, the multilayer film comprises an equal number of layers on either side of the EVOH barrier layer. In some embodiments, there may be a greater number of layers of the film positioned nearer to the cavity of the ostomy pouch than the EVOH barrier layer than further away from the cavity of the pouch than the EVOH barrier layer. In some embodiments, there may be a greater number of layers of the film positioned further away from the cavity of the ostomy pouch than the EVOH barrier layer than nearer to the cavity of the pouch than the EVOH barrier layer.

In some embodiments, at least one tie layer comprises the compatibilizer in a total amount of at least 5 wt. % of the tie layer, or at least 10, 15, or at least 20 wt. % of the tie layer. At least one tie layer may comprise the compatibilizer in a total amount of no greater than 50 wt. % of the tie layer, or no greater than 45, 40, 35, or no greater than 30 wt. % of the tie layer. In some embodiments, at least one tie layer comprises the compatibilizer in a total amount of between 1-50 wt. % of the tie layer, or between 5-30 wt. % of the tie layer, or between 10-30, 15-30, or preferably between 20-30 wt. % of the tie layer.

In some embodiments, at least one compatibilizer comprises a maleic anhydride copolymer. The maleic anhydride copolymer may comprise a maleic anhydride graft copolymer. The maleic anhydride copolymer may comprise a copolymer of maleic anhydride with an olefin. In some embodiments, the olefin is independently chosen from: ethylene, propylene, styrene, and butadiene. In some embodiments, the olefin is independently selected from the group consisting of: ethylene, propylene, styrene, and butadiene. The maleic anhydride copolymer may comprise a copolymer of maleic anhydride with ethylene. In some embodiments, the maleic anhydride copolymer comprises poly(ethylene-graft-maleic anhydride). In preferred embodiments, the maleic anhydride copolymer comprises poly(ethylene-alt-maleic anhydride).

In some embodiments, at least one tie layer may comprise the recycled polymer material in a total amount of at least 40 wt. % of the tie layer, or at least 45, 50, 55, 60, 65, or at least 70 wt. % of the tie layer. At least one tie layer may comprise the recycled polymer material in a total amount of no greater than 95 wt. % of the tie layer, or no greater than 90, 85, 80, or no greater than 75 wt. % of the tie layer. At least one tie layer may comprise the recycled polymer material in a total amount of between 50-100 wt. % of the tie layer, or between 55-95, 60-90, 65-85, or between 70-80 wt. % of the tie layer.

In some preferred embodiments, at least one tie layer comprises the recycled polymer material in a total amount of between 50-100 wt. %, or preferably between 70-80 wt. %. In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein at least one tie layer comprises the recycled polymer in a total amount of between 50-100 wt. %, or preferably between 70-80 wt. %.

In some embodiments, the recycled polymer material comprises EVOH.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises EVOH.

The recycled polymer material may comprise at least one polymer that is independently chosen from: a polyolefin, a polyamide, and combinations thereof. The recycled polymer material may comprise at least one polymer that is independently selected from the group consisting of: a polyolefin, a polyamide, and combinations thereof. In some embodiments, the recycled polymer material comprises a polyolefin. The recycled polymer material may comprise at least one polymer that is independently chosen from: polyethylene, an ethylene copolymer, polypropylene, a propylene copolymer, and combinations thereof. The recycled polymer material may comprise at least one polymer that is independently selected from the group consisting of: polyethylene, an ethylene copolymer, polypropylene, a propylene copolymer, and combinations thereof.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises at least one polymer that is independently selected from the group consisting of: a polyolefin, a polyamide, and combinations thereof.

In some embodiments, the recycled polymer material comprises polyethylene. The recycled polymer material may comprise metallocene-catalysed polyethylene. The recycled polymer material may comprise linear low-density polyethylene. The recycled polymer material may comprise metallocene-catalysed linear low-density polyethylene.

The recycled polymer material may comprise at least one copolymer of ethylene with at least one of: hexene, octene, vinyl acetate, and combinations thereof. The recycled polymer material may comprise ethylene-vinyl acetate (EVA). The EVA may have a vinyl acetate content of between 10-20% of the copolymer, or between 15-20% of the copolymer, or around 18% of the copolymer.

The recycled polymer material may comprise EVOH and at least one polymer that is independently chosen from: a polyolefin, a polyamide, and combinations thereof. The recycled polymer material may comprise EVOH and at least one polymer that is independently selected from the group consisting of: a polyolefin, a polyamide, and combinations thereof. In some embodiments, the recycled polymer material comprises EVOH and a polyolefin. The recycled polymer material may comprise EVOH and at least one polymer that is independently chosen from: polyethylene, an ethylene copolymer, polypropylene, a propylene copolymer, and combinations thereof. The recycled polymer material may comprise EVOH and at least one polymer that is independently selected from the group consisting of: polyethylene, an ethylene copolymer, polypropylene, a propylene copolymer, and combinations thereof.

In some embodiments, the recycled polymer material comprises EVOH and polyethylene. The recycled polymer material may comprise EVOH and metallocene-catalysed polyethylene. The recycled polymer material may comprise EVOH and linear low-density polyethylene. The recycled polymer material may comprise EVOH and metallocene-catalysed linear low-density polyethylene.

The recycled polymer material may comprise EVOH and at least one copolymer of ethylene with at least one of: hexene, octene, vinyl acetate, and combinations thereof. The recycled polymer material may comprise EVOH and ethylene-vinyl acetate (EVA). The EVA may have a vinyl acetate content of between 10-20% of the copolymer, or between 15-20% of the copolymer, or around 18% of the copolymer.

The recycled polymer material may comprise polymer film material, preferably multilayer polymer film material. The multilayer polymer film may comprise at least 2 layers, or at least 3, 4, or at least 5 layers. The multilayer polymer film may comprise 2, 3, 4, 5, 6, 7, 8, 9, 10, or 11 layers. The multilayer polymer film may be the multilayer polymer film of the ostomy pouch of the first aspect of the invention. Statements of invention relating to the multilayer polymer film of the ostomy pouch of the first aspect of the invention may also be applied here.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises polymer film material, preferably multilayer polymer film material.

In some embodiments, the recycled polymer material comprises material from a multilayer polymer film comprising an EVOH barrier layer. The EVOH barrier layer may be as described in statements of invention above for the EVOH barrier layer of the multilayer polymer film of the ostomy pouch of the invention. Such statements of invention above may also be applied here.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises material from a multilayer polymer film comprising an EVOH barrier layer.

In some embodiments, the recycled polymer material comprises material from a multilayer polymer film comprising a polymer carrier layer comprising at least one polymer that is independently chosen from: a polyolefin, a polyamide, and combinations thereof. In some embodiments, the recycled polymer material comprises material from a multilayer polymer film comprising a polymer carrier layer comprising at least one polymer that is independently selected from the group consisting of: a polyolefin, a polyamide, and combinations thereof. The polymer carrier layer may be as described in statements of invention above for the polymer carrier layers of the multilayer polymer film of the ostomy pouch of the invention. Such statements of invention above may also be applied here.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises material from a multilayer polymer film comprising a polymer carrier layer comprising at least one polymer that is independently selected from the group consisting of: a polyolefin, a polyamide, and combinations thereof.

In some embodiments, the recycled polymer material comprises material from a multilayer polymer film comprising a tie layer comprising recycled polymer material and at least one compatibilizer. The tie layer may be as described in statements of invention for the tie layers of the multilayer polymer film of the ostomy pouch of the invention. Such statements of invention may also be applied here.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises material from a multilayer polymer film comprising a tie layer comprising recycled polymer material and at least one compatibilizer.

In some embodiments, the recycled polymer material comprises material from a multilayer polymer film comprising: at least one polymer carrier layer; at least one tie layer comprising polymer material, preferably recycled polymer material, and at least one compatibilizer; and at least one barrier layer comprising EVOH.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises material from a multilayer polymer film comprising: at least one polymer carrier layer; at least one tie layer comprising polymer material, preferably recycled polymer material, and at least one compatibilizer; and at least one barrier layer comprising EVOH.

In some embodiments, the recycled polymer material comprises material from a multilayer polymer film comprising, preferably in order: a first polymer carrier layer; a first tie layer comprising polymer material; a barrier layer comprising EVOH; a second tie layer comprising polymer material; and a second polymer carrier layer. The first and/or second tie layers of the multilayer polymer film may comprise at least one compatibilizer, preferably as described for the multilayer film of the first aspect of the invention. The polymer material in the first and/or second tie layers may comprise recycled polymer material.

In some embodiments, the recycled polymer material comprises unused polymer film material. In some embodiments, the recycled polymer material comprises polymer film edge trim. The recycled polymer material may comprise defective film material.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises unused polymer film material.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises polymer film edge trim.

In some embodiments, the recycled polymer material comprises ostomy film material. The recycled polymer material may comprise ostomy pouch film material, preferably unused ostomy pouch film material. The recycled polymer material may comprise film material of an ostomy pouch of the first aspect of the invention.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises ostomy film material.

In some preferred embodiments, there is provided an ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol) (EVOH); an outer tie layer; and an outer polymer carrier layer; wherein the tie layers each comprise recycled polymer material and at least one compatibilizer, and wherein the recycled polymer material comprises ostomy pouch film material, preferably unused ostomy pouch film material.

In preferred embodiments, the inner and outer tie layers are the same. Alternatively, the inner and outer tie layers may be different.

In some embodiments, at least one tie layer has a thickness of at least 1 micron, or at least 2, 3, 4, 5, or at least 6 microns. At least one tie layer may have a thickness of no greater than 15 microns, or no greater than 14, 13, 12, 11, 10, 9, or no greater than 8 microns. In some embodiments, at least one tie layer has a thickness of between 2-12 microns, or preferably between 4-10 microns, or between 6-8 microns. The inner and/or outer tie layer may have a thickness as described above.

In some embodiments, the multilayer film comprises only 2 tie layers. In some embodiments, the multilayer film comprises 3 or at least 3 tie layers, or 4 or at least 4 tie layers, or 5 or at least 5 tie layers, or 6 or at least 6 tie layers, or 7 or at least 7 tie layers, or 8 or at least 8 tie layers, or 9 or at least 9 tie layers, or 10 or at least 10 tie layers. In preferred embodiments, all tie layers comprise recycled polymer material and at least one compatibilizer, preferably as described in statements of invention above.

The term “inner tie layer” refers to a polymer carrier layer of the multilayer film that is positioned nearer to the cavity of the ostomy pouch than the EVOH barrier layer.

In some embodiments, the multilayer film comprises a single inner tie layer. In other embodiments, the multilayer film comprises a plurality of inner tie layers. The film may comprise 2 or at least 2 inner tie layers, or 3 or at least 3 inner tie layers, or 4 or at least 4 inner tie layers, or 5 or at least 5 inner tie layers.

The multilayer film may comprise a first and second inner tie layer, the second inner tie layer being positioned nearer to the cavity of the ostomy pouch than the first inner tie layer. The first and second inner tie layers may be directly adjacent to each other. Alternatively, the first and second inner tie layers may not be directly adjacent to each other and may be separated by one or more layers of the multilayer film. In some embodiments, the first and second inner polymer carrier layers may be separated by a polymer carrier layer, said polymer carrier layer preferably being as described in statements of invention above.

In some embodiments, the multilayer film may comprise further inner tie layers, such as third, fourth, or fifth inner tie layers, with each consecutively numbered inner tie layer being positioned nearer to the cavity of the pouch than the previously numbered inner tie layer. In some embodiments, at least one inner tie layer is directly adjacent to at least one other inner tie layer. In some embodiments, all inner tie layers are directly adjacent to at least one other inner tie layer. In some embodiments, at least one inner tie layer is not directly adjacent to another inner tie layer, and said inner tie layer may be separated from at least one other inner tie layer by one or more layers of the multilayer film. Said inner tie layer may be separated from at least one other inner tie layer by a polymer carrier layer, said polymer carrier layer preferably being as described in statements of invention above. In some embodiments, all inner tie layers are not directly adjacent to another inner tie layer, and each inner tie layer may be separated from at least one other inner tie layer by one or more layers of the multilayer film. Each inner tie layer may be separated from at least one other inner tie layer by a polymer carrier layer, said polymer carrier layer preferably being as described in statements of invention above.

In preferred embodiments, each inner tie layer comprises recycled polymer material and at least one compatibilizer, preferably as described in statements of invention above.

The term “outer tie layer” refers to a tie layer of the multilayer film that is positioned further away from the cavity of the ostomy pouch than the EVOH barrier layer.

In some embodiments, the multilayer film comprises a single outer tie layer. In other embodiments, the multilayer film comprises a plurality of outer tie layers. The film may comprise 2 or at least 2 outer tie layers, or 3 or at least 3 outer tie layers, or 4 or at least 4 outer tie layers, or 5 or at least 5 outer tie layers.

The multilayer film may comprise a first and second outer tie layer, the second outer tie layer being positioned further away from the cavity of the ostomy pouch than the first outer tie layer. The first and second outer tie layers may be directly adjacent to each other. Alternatively, the first and second outer tie layers may not be directly adjacent to each other and may be separated by one or more layers of the multilayer film. In some embodiments, the first and second outer tie layers may be separated by a polymer carrier layer, said polymer carrier layer preferably being as described in statements of invention above.

In some embodiments, the multilayer film may comprise further outer tie layers, such as third, fourth, or fifth outer tie layers, with each consecutively numbered outer tie layer being positioned further away from the cavity of the pouch than the previously numbered outer tie layer. In some embodiments, at least one outer tie layer is directly adjacent to at least one other outer tie layer. In some embodiments, all outer tie layers are directly adjacent to at least one other outer tie layer. In some embodiments, at least one outer tie layer is not directly adjacent to another outer tie layer, and said outer tie layer may be separated from at least one other outer tie layer by one or more layers of the multilayer film. Said outer tie layer may be separated from at least one other outer tie layer by a polymer carrier layer, said polymer carrier layer preferably being as described in statements of invention above. In some embodiments, all outer tie layers are not directly adjacent to another outer tie layer, and each outer tie layer may be separated from at least one other outer tie layer by one or more layers of the multilayer film. Each outer tie layer may be separated from at least one other outer tie layer by a polymer carrier layer, said polymer carrier layer preferably being as described in statements of invention above.

In preferred embodiments, each outer tie layer comprises recycled polymer material and at least one compatibilizer.

In some embodiments, the multilayer film comprises a greater number of outer tie layers than inner tie layers. In some embodiments, the multilayer film comprises a greater number of inner tie layers than outer tie layers. In some preferred embodiments, the multilayer film comprises the same number of outer tie layers as inner tie layers.

In some preferred embodiments, the multilayer film comprises the same number of polymer carrier layers as tie layers. In some embodiments, the multilayer film comprises the same number of polymer carrier layers as tie layers comprising recycled polymer material and at least one compatibilizer. In other embodiments, the multilayer film comprises a greater number of polymer carrier layers than tie layers. In other embodiments, the multilayer film comprises a greater number of tie layers than polymer carrier layers.

In some embodiments, tie layers comprising recycled polymer material and at least one compatibilizer are used to adhere polymer carrier layers to each other, to the EVOH barrier layer, and/or to a weld layer as described below.

In some embodiments, at least one or each tie layer comprises recycled polymer material and a compatibilizer comprising a maleic anhydride copolymer, preferably a copolymer of maleic anhydride with ethylene, preferably poly (ethylene-alt-maleic anhydride); and at least one or each polymer carrier layer comprises at least one polymer that is independently chosen from: polyethylene, an ethylene copolymer, polypropylene, a propylene copolymer, and combinations thereof. In some embodiments, at least one or each tie layer comprises recycled polymer material and a compatibilizer comprising a maleic anhydride copolymer, preferably a copolymer of maleic anhydride with ethylene, preferably poly (ethylene-alt-maleic anhydride); and at least one or each polymer carrier layer comprises at least one polymer that is independently selected from the group consisting of: polyethylene, an ethylene copolymer, polypropylene, a propylene copolymer, and combinations thereof.

In some embodiments, the multilayer barrier film further comprises an outermost weld layer positioned furthest from the pouch cavity. In some embodiments, the outermost weld layer is positioned adjacent to the layer of the multilayer film that is positioned furthest from the pouch cavity. In some embodiments, the outermost weld layer may be positioned adjacent to the polymer carrier layer that is positioned furthest from the pouch cavity. In some embodiments, the outermost weld layer may be positioned adjacent to the tie layer that is positioned furthest from the pouch cavity.

In some embodiments, the multilayer barrier film further comprises an innermost weld layer positioned nearest the pouch cavity. In some embodiments, the innermost weld layer is positioned adjacent to the layer of the multilayer film that is positioned nearest the pouch cavity. In some embodiments, the innermost weld layer may be positioned adjacent to the polymer carrier layer that is positioned nearest the pouch cavity. In some embodiments, the innermost weld layer may be positioned adjacent to the tie layer that is positioned nearest the pouch cavity.

In some embodiments, the multilayer film comprises both an innermost and outermost weld layer.

At least one weld layer may comprise at least one chemically inert polymer. In some embodiments, the chemically inert polymer may be present in a total amount of at least 80 wt. % of the weld layer, or at least 85, or at least 90 wt. % of the weld layer. The weld layer may comprise the chemically inert polymer in a total amount of between 80-100 wt. % of the weld layer, or between 85-100, 90-100, or between 90-95 wt. % of the weld layer.

The innermost and/or outermost weld layer may comprise at least one polymer that is independently chosen from: a polyolefin, a polyamide, and combinations thereof. The innermost and/or outermost weld layer may comprise at least one polymer that is independently selected from the group consisting of: a polyolefin, a polyamide, and combinations thereof. The innermost and/or outermost weld layer may comprise a polyolefin. The innermost and/or outermost weld layer may comprise at least one polymer that is independently chosen from: polyethylene, an ethylene copolymer, polypropylene, a propylene copolymer, and combinations thereof. The innermost and/or outermost weld layer may comprise at least one polymer that is independently selected from the group consisting of: polyethylene, an ethylene copolymer, polypropylene, a propylene copolymer, and combinations thereof. In such embodiments, the polymer may be present in a total amount as described for the chemically inert polymer above.

The innermost and/or outermost weld layer may comprise at least one copolymer of ethylene with at least one of: hexene, octene, vinyl acetate, and combinations thereof. The innermost and/or outermost weld layer may preferably comprise ethylene-vinyl acetate (EVA). The EVA may have a vinyl acetate content of between 10-20% of the copolymer, or around 18% of the copolymer. In such embodiments, the copolymer may be present in a total amount as described for the chemically inert polymer above.

In some embodiments, the innermost and outermost weld layers comprise the same polymer. In preferred embodiments, the innermost and outermost weld layers both comprise EVA. In other embodiments, the innermost and outermost weld layers comprise different polymers.

The innermost and/or outermost weld layer may have a thickness of at least 2 microns, or at least 3, 4, 5, 6, 7, 8, 9, or at least 10 microns. The innermost and/or outermost weld layer may have a thickness of no greater than 25 microns, or no greater than 20, or no greater than 15 microns. The innermost and/or outermost weld layer may have a thickness of between 5-15 microns, or between 7-13, or between 9-13 microns. In preferred embodiments, the innermost and outermost weld layers comprise the same thickness.

The innermost and/or outermost weld layer may further comprise a slip agent. The slip agent may be present in a total amount of between 0.1-5 wt. % of the weld layer, or between 0.1-2 wt. %, or between 0.1-1 wt. % of the weld layer.

The layers of the multilayer film may be held together by at least one bond formed between the innermost and outermost weld layers. The multilayer film may be held together by at least one weld formed between the innermost and outermost weld layers. The weld may pass through the layers of the multilayer film held between the innermost and outermost weld layers. Alternatively, the weld may only be between the innermost and outermost weld layers and may not pass through the layers of the multilayer film held between the weld layers. In such embodiments, the innermost and outermost weld layers may extend beyond the perimeters of the layers of the film held between the weld layers, and the weld between the innermost and outermost weld layers may be positioned beyond the perimeters of the layers of the film held between the weld layers.

In preferred embodiments, the multilayer film is a cast film. In other embodiments, the film may be a blown film.

In some embodiments, the multilayer film has a thickness of at least 30 microns, or at least 35, 40, 45, 50, 55, or at least 60 microns. The multilayer film may have a thickness of no greater than 140 microns, or no greater than 130, 120, 110, 100, 90, or no greater than 80 microns. In some embodiments, the multilayer film has a thickness of between 40-130 microns, or between 50-120, or between 60-110 microns. In some embodiments, the multilayer film has a thickness of between 50-90 microns, or between 55-85, or between 60-80 microns.

In some embodiments, at least one layer of the multilayer film further comprises an anti-block agent. The anti-block agent may be present in a total amount of between 0.1-10 wt. % of the layer, or between 0.5-8 wt. % of the layer. In some embodiments, at least one polymer carrier layer and/or weld layer may comprise the anti-block agent. In some embodiments, the outermost and/or innermost weld layer may comprise the anti-block agent.

In some preferred embodiments, the multilayer film has five layers in total or at least 5 layers, the five layers comprising in order from adjacent to the cavity outwards: an inner polymer carrier layer; an inner tie layer comprising recycled polymer material and at least one compatibilizer; a barrier layer comprising EVOH; an outer tie layer comprising recycled polymer material and at least one compatibilizer; and an outer polymer carrier layer.

In such preferred embodiments, the inner polymer carrier may preferably be as described in statements of invention above. In such preferred embodiments, the inner polymer carrier layer may comprise a polyolefin, preferably polyethylene. The inner polymer carrier layer may comprise metallocene-catalysed polyethylene. The inner polymer carrier layer may comprise linear low-density polyethylene, preferably metallocene-catalysed linear low-density polyethylene.

In such preferred embodiments, the inner tie layer may preferably be as described in statements of invention above. In such preferred embodiments, the inner tie layer may comprise a maleic anhydride copolymer compatibilizer, preferably a copolymer of maleic anhydride with ethylene, more preferably poly (ethylene-alt-maleic anhydride). The inner tie layer may preferably further comprise recycled polymer material from a multilayer polymer film comprising: an EVOH barrier layer, and a polymer carrier layer comprising at least one polymer that is independently chosen from: a polyolefin, a polyamide, and combinations thereof. The inner tie layer may preferably further comprise recycled polymer material from a multilayer polymer film comprising: an EVOH barrier layer, and a polymer carrier layer comprising at least one polymer that is independently selected from the group consisting of: a polyolefin, a polyamide, and combinations thereof.

In such preferred embodiments, the EVOH barrier layer may preferably be as described in statements of invention above. In some embodiments, the EVOH comprises between 10-50% vinyl alcohol units, or between 15-45%, or between 20-40%, or between 25-35%, or between 30-35% vinyl alcohol units.

In such preferred embodiments, the outer tie layer may preferably be as described in statements of invention above. Statements above relating to the inner tie layer may also be applied to the outer tie layer.

In such preferred embodiments, the outer polymer carrier layer may preferably be as described in statements of invention above. In such preferred embodiments, the inner polymer carrier layer may comprise a polyolefin, preferably a polyethylene copolymer. The outer polymer carrier layer may comprise at least one copolymer of ethylene with at least one of: hexene, octene, vinyl acetate, and combinations thereof. Preferably, the outer polymer carrier layer comprises EVA.

In some embodiments, the multilayer film has 6 layers in total or at least 6 layers. The 6 layers may comprise the five layers of the 5-layer film described above and an outermost or innermost weld layer.

In some preferred embodiments, the multilayer film has 7 layers in total or at least 7 layers. The 7 layers may comprise the five layers of the 5-layer film described above, and an innermost and outermost weld layer.

In such preferred embodiments, the outermost and/or innermost weld layer may preferably be as described in statements of invention above. In such preferred embodiments, the outermost and/or innermost weld layer may comprise a polyolefin, preferably a polyethylene copolymer. The outermost and/or innermost weld layer may comprise at least one copolymer of ethylene with at least one of: hexene, octene, vinyl acetate, and combinations thereof. Preferably, the outermost and/or innermost weld layer comprises EVA.

In some embodiments, the multilayer film may have 8 or at least 8 layers, or 9 or at least 9 layers, or 10 or at least 10 layers. Multilayer films with 8 or more layers may comprise the 7 layers as described for the 7-layer film above, and may comprise at least one further layer that is independently chosen from: a polymer carrier layer; a tie layer, preferably comprising recycled polymer material and at least one compatibilizer; and combinations thereof. Multilayer films with 8 or more layers may comprise the 7 layers as described for the 7-layer film above, and may comprise at least one further layer that is independently selected from the group consisting of: a polymer carrier layer; a tie layer, preferably comprising recycled polymer material and at least one compatibilizer; and combinations thereof. In some embodiments, the multilayer film may comprise further polymer carrier layers and tie layers, and the polymer carrier and tie layers may be arranged alternately in the film.

In some embodiments, the ostomy pouch comprises a front wall and a rear wall, wherein the front and rear walls are sealed together to form the cavity of the pouch therebetween, and wherein the front and rear wall comprise the multilayer barrier film.

In some embodiments, the front and rear walls are sealed together at their peripheries to form the cavity. The front and rear walls may be welded together at their peripheries to form the cavity.

In some embodiments, the outermost and/or innermost weld layer of the film of the front wall may be welded to the outermost and/or innermost weld layer of the film of the rear wall. In some embodiments, the outermost weld layer of the film of the front wall may be welded to the outermost weld layer of the film of the rear wall. In some embodiments, the weld may pass through the other layers of the multilayer film of the front and/or rear wall. The weld may pass through the layers of the multilayer film held between the innermost and outermost weld layers of the front and/or rear wall. Alternatively, the weld may only be between the innermost and/or outermost weld layers of the films of the front and rear walls and may not pass through the layers of the multilayer films held between the weld layers. In such embodiments, the innermost and/or outermost weld layers may extend beyond the perimeters of the layers of the film held between the weld layers, and the weld between the innermost and/or outermost weld layers of the films of the front and rear walls may be positioned beyond the perimeters of the layers of the film held between the weld layers.

In some embodiments, the rear wall comprises an inlet for receiving human waste.

In some embodiments, the front wall comprises a filter for deodorising odorous gas from the human waste. The filter may comprise an opening for emitting deodorised gas into the atmosphere.

In some embodiments, the ostomy pouch comprises an attachment element for attaching the pouch to the body of a patient about a stoma. In some embodiments, the attachment element comprises a mechanical fastening means. In some embodiments, the attachment element comprises an adhesive element configured to adhere the pouch to the skin of the patient surrounding the stoma. In some embodiments, the attachment element comprises an adhesive flange for removably adhering the pouch to the body of a patient. In some embodiments, the attachment element comprises an elastic band or strap that can be stretched and secured around the body of the patient. In some embodiments, the attachment element comprises a belt or waistband adapted to be worn around the waist of the patient.

According to a second aspect of the invention, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b).

The ostomy pouch of the second aspect of the invention is preferably the ostomy pouch of the first aspect of the invention. The multilayer barrier film of the second aspect of the invention is preferably the multilayer barrier film of the first aspect of the invention. Components of the ostomy pouch or barrier film of the second aspect of the invention are preferably as described for the first aspect of the invention above. The first polymer carrier layer of the film of the second aspect of the invention is preferably the inner polymer carrier layer of the film of the first aspect of the invention. The first tie layer of the film of the second aspect of the invention is preferably the inner tie layer of the film of the first aspect of the invention. The barrier layer of the film of the second aspect of the invention is preferably the barrier layer of the film of the first aspect of the invention. The second tie layer of the film of the second aspect of the invention is preferably the outer tie layer of the film of the first aspect of the invention. The second polymer carrier layer of the film of the second aspect of the invention is preferably the outer polymer carrier layer of the film of the first aspect of the invention.

Statements of invention for the first aspect of the invention above may also be applied mutatis mutandis to the second aspect of the invention. Statements of invention below for the second aspect of the invention may also be applied mutatis mutandis to the other aspects of the invention.

The method may comprise a further step prior to step (a) of forming the front wall. Forming the front wall may comprise: providing a first and second carrier layer polymer composition; providing a barrier layer polymer composition comprising EVOH; and providing a first and second tie layer polymer composition by combining recycled polymer material and at least one compatibilizer; and co-extruding the first and second carrier layer, barrier layer, and first and second tie layer polymer compositions to provide the multilayer barrier film of the front wall, such that the film comprises in order: the first polymer carrier layer; the first tie layer; the barrier layer; the second tie layer; and the second polymer carrier layer.

The method may comprise a further step prior to step (a) of forming the rear wall. Forming the rear wall may comprise: providing a first and second carrier layer polymer composition; providing a barrier layer polymer composition comprising EVOH; and providing a first and second tie layer polymer composition by combining recycled polymer material and at least one compatibilizer; and co-extruding the first and second carrier layer, barrier layer, and first and second tie layer polymer compositions to provide the multilayer barrier film of the rear wall, such that the film comprises in order: the first polymer carrier layer; the first tie layer; the barrier layer; the second tie layer; and the second polymer carrier layer.

The method may further comprise the step before step (a) of manufacturing the multilayer barrier film of the front and/or rear walls. Manufacturing the multilayer film may comprise the steps of: providing a first and second carrier layer polymer composition; providing a barrier layer polymer composition comprising EVOH; providing a first and second tie layer polymer composition by combining recycled polymer material and at least one compatibilizer; and co-extruding the first and second carrier layer, barrier layer, and first and second tie layer polymer compositions to provide the multilayer barrier film. The polymer compositions may preferably comprise polymer melts. Alternatively, the polymer compositions may comprise solid polymer sheets or pellets. The extrusion method may comprise multilayer cast extrusion.

The method may comprise the further step of forming the first and/or second tie layer polymer composition. Forming the first and/or second tie layer polymer composition may comprise combining at least one compatibilizer with recycled polymer material. The compatibilizer may preferably be as described in statements of invention for the first aspect of the invention above. The recycled polymer material may preferably be as described in statements of invention above. The recycled polymer material may comprise polymer film material, preferably as described for the first aspect of the invention above. The recycled polymer material may comprise material from a multilayer polymer film, preferably as described for the first aspect of the invention. The recycled polymer material may comprise unused polymer film material. The recycled polymer material may comprise polymer film edge trim. The recycled polymer film material may comprise defective film material. The recycled polymer material may comprise ostomy film material.

In some embodiments, forming the first and/or second tie layer comprises the step of providing the recycled polymer material. The step of providing the recycled polymer material may comprise: providing polymer film material, preferably multilayer polymer film material as described above; converting the polymer film material into flakes; and optionally converting the flakes into pellets.

In some embodiments, the method of the second aspect of the invention is repeated at least once. In at least one repeated run of the method, the recycled polymer material may comprise unused polymer film material, such as polymer film edge trim from a previous run of the method of manufacturing an ostomy pouch of the second aspect of the invention. The method may comprise continuously transferring unused polymer film material from a previous run of the method, preferably edge trim or defective film material directly back into a feed stream for use in further repetitions of the method. The method may comprise grinding and granulating unused polymer film from a previous run of the method, preferably edge trim or defective film material, to provide the recycled polymer material.

Step (b) may comprise welding the front and rear walls together, preferably as described for the first aspect of the invention above.

The welding may employ a method that is independently chosen from: ultrasonic welding, heat sealing, radio frequency welding, laser welding, and combinations thereof. The welding may employ a method that is independently selected from the group consisting of: ultrasonic welding, heat sealing, radio frequency welding, laser welding, and combinations thereof.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises EVOH.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises at least one polymer that is independently selected from the group consisting of: a polyolefin, a polyamide, and combinations thereof.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises polymer film material, preferably multilayer polymer film material.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises material from a multilayer polymer film comprising an EVOH barrier layer.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises material from a multilayer polymer film comprising a polymer carrier layer comprising at least one polymer that is independently selected from the group consisting of: a polyolefin, a polyamide, and combinations thereof.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises unused polymer film material.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises polymer film edge trim.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises ostomy film material.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises ostomy pouch film material, preferably unused ostomy pouch film material.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein at least one tie layer comprises the recycled polymer material in a total amount of between 50-100 wt. %, or preferably between 70-80 wt. %.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises material from a multilayer polymer film comprising a tie layer comprising recycled polymer material and at least one compatibilizer.

In some preferred embodiments, there is provided a method of manufacturing an ostomy pouch comprising the steps of:
(a) Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol) (EVOH); a second tie layer comprising recycled polymer material and at least one compatibilizer; and a second polymer carrier layer; and
(b) Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b), and wherein the recycled polymer material comprises material from a multilayer polymer film comprising: at least one polymer carrier layer; at least one tie layer comprising recycled polymer material and at least one compatibilizer; and at least one barrier layer comprising EVOH.

According to a third aspect of the invention, there is provided a multilayer barrier film comprising in order: a first polymer carrier layer; a first tie layer; a barrier layer comprising poly(ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer.

The multilayer barrier film of the third aspect of the invention is preferably the multilayer barrier film of the first aspect of the invention. Components of the barrier film of the third aspect of the invention are preferably as described for the first aspect of the invention above. The first polymer carrier layer of the film of the third aspect of the invention is preferably the inner polymer carrier layer of the film of the first aspect of the invention. The first tie layer of the film of the third aspect of the invention is preferably the inner tie layer of the film of the first aspect of the invention. The barrier layer of the film of the third aspect of the invention is preferably the barrier layer of the film of the first aspect of the invention. The second tie layer of the film of the third aspect of the invention is preferably the outer tie layer of the film of the first aspect of the invention. The second polymer carrier layer of the film of the third aspect of the invention is preferably the outer polymer carrier layer of the film of the first aspect of the invention.

Statements of invention for the first and second aspects of the invention above may also be applied mutatis mutandis to the third aspect of the invention. Statements of invention below for the third aspect of the invention may also be applied mutatis mutandis to the other aspects of the invention.

In some embodiments, the compatibilizer may be present in a total amount of between 10-30 wt. % of each tie layer, or between 15-30, or preferably between 20-30 wt. % of each tie layer.

In preferred embodiments, the first tie layer may comprise the compatibilizer in the same amount as the second tie layer.

In some embodiments, at least one compatibilizer comprises a maleic anhydride copolymer. The maleic anhydride copolymer may comprise a maleic anhydride graft copolymer. The maleic anhydride copolymer may comprise a copolymer of maleic anhydride with an olefin. In some embodiments, the olefin is independently chosen from: ethylene, propylene, styrene, and butadiene. In some embodiments, the olefin is independently selected from the group consisting of: ethylene, propylene, styrene, and butadiene. The maleic anhydride copolymer may comprise a copolymer of maleic anhydride with ethylene. In some embodiments, the maleic anhydride copolymer comprises poly(ethylene-graft-maleic anhydride). In preferred embodiments, the maleic anhydride copolymer comprises poly(ethylene-alt-maleic anhydride).

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises EVOH.

In some preferred embodiments, there is provided a multilayer barrier film comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises at least one polymer that is independently selected from the group consisting of: a polyolefin, a polyamide, and combinations thereof.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises polymer film material, preferably multilayer polymer film material.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises material from a multilayer polymer film comprising an EVOH barrier layer.

In some preferred embodiments, there is provided a multilayer barrier film comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises material from a multilayer polymer film comprising a polymer carrier layer comprising at least one polymer that is independently selected from the group consisting of: a polyolefin, a polyamide, and combinations thereof.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises unused polymer film material.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises polymer film edge trim.

In some preferred embodiments, there is provided a multilayer barrier film comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises ostomy film material.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises ostomy pouch film material, preferably unused ostomy pouch film material.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein at least one tie layer comprises the recycled polymer material in a total amount of between 50-100 wt. %, or preferably between 70-80 wt. %.

In some preferred embodiments, there is provided a multilayer barrier film comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises material from a multilayer polymer film comprising a tie layer comprising recycled polymer material and at least one compatibilizer.

In some preferred embodiments, there is provided a multilayer barrier film comprising in order: a first polymer carrier layer; a first tie layer; a barrier layer comprising poly (ethylene-co-vinyl alcohol) (EVOH); a second tie layer; and a second polymer carrier layer; wherein the tie layers each comprise recycled polymer material and at least one compatibilizer, the at least one compatibilizer being present in a total amount of between 5-30 wt. % of each tie layer, and wherein the recycled polymer material comprises material from a multilayer polymer film comprising: at least one polymer carrier layer; at least one tie layer comprising recycled polymer material and at least one compatibilizer; and at least one barrier layer comprising EVOH.

According to a fourth aspect of the invention, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

The multilayer barrier film of the fourth aspect of the invention is preferably the multilayer barrier film of the third aspect of the invention. Components of the barrier film of the fourth aspect of the invention are preferably as described for the third aspect of the invention above. The first polymer carrier layer of the film of the fourth aspect of the invention is preferably the inner polymer carrier layer of the film of the first aspect of the invention. The first tie layer of the film of the fourth aspect of the invention is preferably the inner tie layer of the film of the first aspect of the invention. The barrier layer of the film of the fourth aspect of the invention is preferably the barrier layer of the film of the first aspect of the invention. The second tie layer of the film of the fourth aspect of the invention is preferably the outer tie layer of the film of the first aspect of the invention. The second polymer carrier layer of the film of the fourth aspect of the invention is preferably the outer polymer carrier layer of the film of the first aspect of the invention.

Statements of invention for the other aspects of the invention above may also be applied mutatis mutandis to the fourth aspect of the invention. Statements of invention below for the fourth aspect of the invention may also be applied mutatis mutandis to the other aspects of the invention.

The polymer compositions may preferably comprise polymer melts. Alternatively, the polymer compositions may comprise solid polymer sheets or pellets.

The method of forming the first and/or second tie layer polymer composition may be as described for the second aspect of the invention above. Statements of invention for the second aspect of the invention may also be applied here.

Step (d) may comprise multilayer cast extrusion.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising EVOH; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising at least one polymer that is independently selected from the group consisting of: a polyolefin, a polyamide, and combinations thereof; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising polymer film material, preferably multilayer polymer film material; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising material from a multilayer polymer film comprising an EVOH barrier layer; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising material from a multilayer polymer film comprising a polymer carrier layer comprising at least one polymer that is independently selected from the group consisting of: a polyolefin, a polyamide, and combinations thereof; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising unused polymer film material; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising polymer film edge trim; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising ostomy film material; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising ostomy pouch film material, preferably unused ostomy pouch film material; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and at least one tie layer composition comprising the recycled polymer material in a total amount of between 50-100 wt. %, or preferably between 70-80 wt. %; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising material from a multilayer polymer film comprising a tie layer comprising recycled polymer material and at least one compatibilizer; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

In some preferred embodiments, there is provided a method of manufacturing a multilayer barrier film comprising the steps of:
(a) Providing a first and second carrier layer polymer composition;
(b) Providing a barrier layer polymer composition comprising poly(ethylene-co-vinyl alcohol) (EVOH);
(c) Providing a first and second tie layer polymer composition by combining recycled polymer material and at least one compatibilizer, the at least one compatibilizer being present in a total amount of between 5-30 wt. % of each tie layer polymer composition, and the recycled polymer material comprising material from a multilayer polymer film comprising: at least one polymer carrier layer; at least one tie layer comprising recycled polymer material and at least one compatibilizer; and at least one barrier layer comprising EVOH; and
(d) Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.

### DETAILED DESCRIPTION OF THE INVENTION

In order that the invention may be more clearly understood embodiments thereof will now be described, by way of example only, with reference to the accompanying drawings, of which:

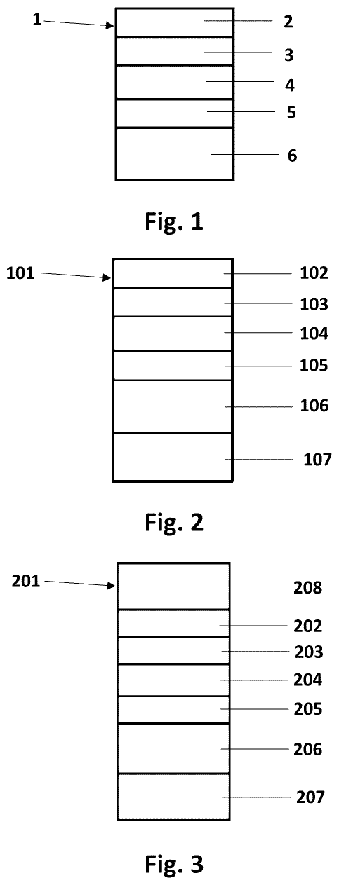

FIG. 1 shows a cross-sectional view of an embodiment of a 5-layer cast film of the invention for use in an ostomy pouch of the invention.

FIG. 2 shows a cross-sectional view of an embodiment of a 6-layer cast film of the invention for use in an ostomy pouch of the invention.

FIG. 3 shows a cross-sectional view of an embodiment of a 7-layer cast film of the invention for use in an ostomy pouch of the invention.

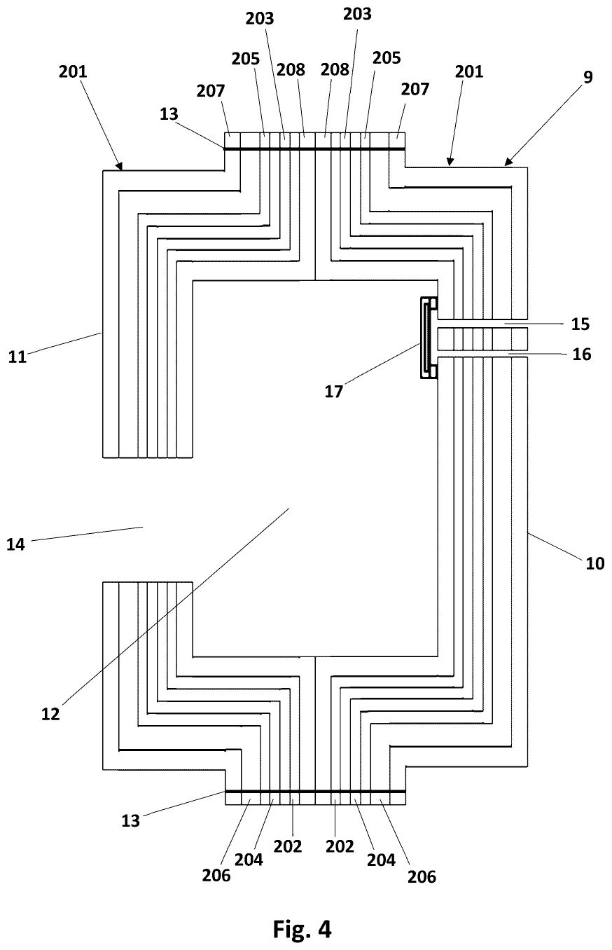

FIG. 4 shows a side cross-sectional view of an embodiment of an ostomy pouch of the invention comprising the 7-layer cast film of FIG. 3.

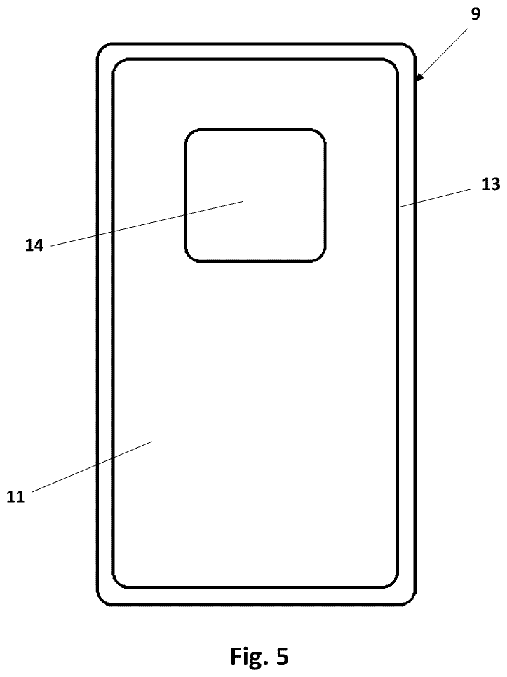

FIG. 5 shows a top-down view of the ostomy pouch of FIG. 4 showing the rear wall of the pouch.

### Example 1: 5-Layer Cast Film of the Invention

FIG. 1 illustrates an embodiment of a 5-layer cast film 1 of the invention for use in an ostomy pouch of the invention.

The 5-layer cast film 1 has a layer structure as described in Table 1 below. The order of arrangement of the layers of the film 1 are as depicted in Table 1.

TABLE 1

| | | | Layer thickness |
|---|---|---|---|
| Layer No. | Layer type | Layer materials | (μm) |
| 2 | First polymer | Metallocene- | 7.5 |
| | carrier layer | catalysed linear | |
| | | low-density | |
| | | polyethylene: | |
| | | 92.5% | |
| | | Pigment: 7% | |
| 3 | First tie layer | Recycled polymer | 7.5 |
| | | material: 75% | |
| | | Poly(ethylene-alt- | |
| | | maleic anhydride) | |
| | | compatibilizer: | |
| | | 25% | |
| 4 | Barrier layer | EVOH (32%): | 8.3 |
| | | 100% | |
| 5 | Second tie layer | Recycled polymer | 7.5 |
| | | material: 75% | |
| | | Poly(ethylene-alt- | |
| | | maleic anhydride) | |
| | | compatibilizer: | |
| | | 25% | |
| 6 | Second polymer | EVA (18%): 93% | 14.4 |
| | carrier layer | Pigment: 7% | |

The 5-layer film 1 is produced my multilayer cast extrusion, which involves providing in polymer melt form: first and second carrier layer polymer compositions corresponding to carrier layers 2 and 6; a barrier layer polymer composition corresponding to barrier layer 4; and first and second tie layer polymer compositions corresponding to tie layers 3 and 5. The polymer compositions for the layers of the film are then co-extruded to form the multilayer film 1.

The recycled polymer film material present in tie layers 3 and 5 comprises unused polymer film material (edge trim and defective film material) from previous productions of 5-layer film 1. The unused polymer film material from previous productions is ground, granulated, combined with compatibilizer to form a tie layer polymer composition; and re-casted to form the new film.

Film 1 allows for highly reduced odour permeability, in particular of the main faecal odorant, hydrogen sulphide. Further, the film 1 is fully recyclable and so allows for scrap film material generated in the construction of new films to be reused in the manufacture of other films, thus leading to significant cost and material savings in film manufacture. The film 1 also advantageously provides excellent optimal and mechanical properties suited for ostomy applications.

### Example 2: 6-Layer Cast Film of the Invention

FIG. 2 illustrates an embodiment of a 6-layer cast film 101 of the invention for use in an ostomy pouch of the invention.

The 6-layer cast film 101 has a layer structure as described in Table 2 below. The order of arrangement of the layers of the film 101 are as depicted in Table 2.

TABLE 2

| | | | Layer thickness |
|---|---|---|---|
| Layer No. | Layer type | Layer materials | (μm) |
| 102 | First polymer | Metallocene- | 7.5 |
| | carrier layer | catalysed linear | |
| | | low-density | |
| | | polyethylene: | |
| | | 92.5% | |
| | | Pigment: 7% | |
| 103 | First tie layer | Recycled polymer | 7.5 |
| | | material: 75% | |
| | | Poly(ethylene-alt- | |
| | | maleic anhydride) | |
| | | compatibilizer: | |
| | | 25% | |
| 104 | Barrier layer | EVOH (32%): | 8.3 |
| | | 100% | |
| 105 | Second tie layer | Recycled polymer | 7.5 |
| | | material: 75% | |
| | | Poly(ethylene-alt- | |
| | | maleic anhydride) | |
| | | compatibilizer: | |
| | | 25% | |
| 106 | Second polymer | EVA (18%): 93% | 14.4 |
| | carrier layer | Pigment: 7% | |
| 107 | Weld layer (outer) | EVA (18%): 92.5% | 11.5 |
| | | Anti-block agent: | |
| | | 7% | |
| | | Slip agent: 0.5% | |

The 6-layer film 101 is produced my multilayer cast extrusion, which involves providing in polymer melt form: first and second carrier layer polymer compositions corresponding to carrier layers 102 and 106; a barrier layer polymer composition corresponding to barrier layer 104; first and second tie layer polymer compositions corresponding to tie layers 103 and 105; and a weld layer polymer composition corresponding to outer weld layer 107. The polymer compositions for the layers of the film are then co-extruded to form the multilayer film 101.

The recycled polymer film material present in tie layers 103 and 105 comprises unused polymer film material (edge trim and defective film material) from previous productions of 6-layer film 101. The unused polymer film material from previous productions is ground, granulated, combined with compatibilizer to form a tie layer polymer composition; and re-casted to form the new film.

As for 5-layer film 1, 6-layer film 101 also provided numerous advantages relating to odour permeability, recyclability, and film optical and mechanical properties.

### Example 3: 7-Layer Cast Film of the Invention

FIG. 3 illustrates an embodiment of a 7-layer cast film 201 of the invention for use in an ostomy pouch of the invention.

The 7-layer cast film 201 has a layer structure as described in Table 3 below. The order of arrangement of the layers of the film 201 are as depicted in Table 3.

TABLE 3

| | | | Layer thickness |
|---|---|---|---|
| Layer No. | Layer type | Layer materials | (μm) |
| 208 | Weld layer (inner) | EVA (18%): 93% | 11.5 |
| | | Anti-block agent: | |
| | | 7% | |
| 202 | First polymer | Metallocene- | 7.5 |
| | carrier layer | catalysed linear | |
| | | low-density | |
| | | polyethylene: | |
| | | 92.5% | |
| | | Pigment: 7% | |
| 203 | First tie layer | Recycled polymer | 7.5 |
| | | material: 75% | |
| | | Poly(ethylene-alt- | |
| | | maleic anhydride) | |
| | | compatibilizer: | |
| | | 25% | |
| 204 | Barrier layer | EVOH (32%): | 8.3 |
| | | 100% | |
| 205 | Second tie layer | Recycled polymer | 7.5 |
| | | material: 75% | |
| | | Poly(ethylene-alt- | |
| | | maleic anhydride) | |
| | | compatibilizer: | |
| | | 25% | |
| 206 | Second polymer | EVA (18%): 93% | 14.4 |
| | carrier layer | Pigment: 7% | |
| 207 | Weld layer (outer) | EVA (18%): 93% | 11.5 |
| | | Anti-block agent: | |
| | | 7% | |

The 7-layer film 201 is produced my multilayer cast extrusion, which involves providing in polymer melt form: first and second carrier layer polymer compositions corresponding to carrier layers 202 and 206; a barrier layer polymer composition corresponding to barrier layer 204; first and second tie layer polymer compositions corresponding to tie layers 203 and 205; and a first and second weld layer polymer composition corresponding to outer weld layer 207 and inner weld layer 208. The polymer compositions for the layers of the film are then co-extruded to form the multilayer film 201.

The recycled polymer film material present in tie layers 203 and 205 comprises unused polymer film material (edge trim and defective film material) from previous productions of 7-layer film 201. The unused polymer film material from previous productions is ground, granulated, combined with compatibilizer to form a tie layer polymer composition; and re-casted to form the new film.

As for 5-layer film 1, 7-layer film 201 also provided numerous advantages relating to odour permeability, recyclability, and film optical and mechanical properties.

### Example 4: Ostomy Pouch of the Invention Comprising 7-Layer Cast Film of the Invention

FIGS. 4 and 5 illustrate an embodiment of an ostomy pouch 9 of the invention comprising 7-layer cast film 201 of the invention.

The pouch 9 comprises a front wall 10 and a rear wall 11. Both the front wall 10 and rear wall 11 comprise a 7-layer cast film 201 of the invention, as described in Example 3 above. The front wall 10 and rear wall 11 are sealed together to form a cavity 12 of the pouch 9 therebetween. The front wall 10 and rear wall 11 are sealed together such that inner weld layers 208 of both the front wall 10 and rear wall 11 are arranged nearest to the cavity 12 of the pouch 9 and the outer weld layers 207 of both the front wall 10 and rear wall 11 are arranged furthest away from the cavity 12 of the pouch 9. As shown in FIG. 5, the front wall 10 and rear wall 11 are sealed together by a weld 13 formed at their peripheries to form the cavity 12. The weld passes through all layers of the front wall 10 and rear wall 11 in order to hold the front wall 10 and rear wall 11 together.

The rear wall 11 of the pouch 9 further comprises an inlet 14 for receiving human waste which comprises an aperture formed in the rear wall 11.

As shown in FIG. 4, the front wall 10 of the pouch further comprises two venting apertures 15 and 16 formed in the front wall 10. The front wall 10 further comprises a filter 17 for deodorising odorous gas from human waste secured to the first polymer carrier layer 102 of the film 201 of the front wall 10, such that the filter 17 covers vent apertures 15 and 16. In other embodiments of ostomy pouches of the invention, the pouch may not comprise a filter and may be a non-filter bearing pouch.

The ostomy pouch 9 is used in the conventional manner. The rear wall 11 further comprises an attachment (not shown) for attaching the pouch 9 to the body of a patient about a stoma.

Ostomy pouch 9 displays excellent odour barrier properties through superior odour impermeability, particularly to hydrogen sulphide odour. The pouch 9 is also fully recyclable and thus highly environmentally friendly. Further, the 7-layer cast film 201 employed in the pouch 9 provides the pouch 9 with excellent optical and mechanical properties that are especially suited to ostomy applications.

### Example 5: Determination of Hydrogen Sulphide Permeation of Multilayer Films of the Invention

To investigate the odour impermeability of multilayer films of the invention further, tests were performed to evaluate the permeation (P) of hydrogen sulphide through films of the invention and compare performance to that of multilayer films not of the invention.

Results of the tests are displayed in Table 4 below.

TABLE 4

| | | | P, hydrogen |
|---|---|---|---|
| | | | sulphide (Ncm³ |
| | Barrier layer type | Film thickness | mm m⁻²d⁻¹bar⁻¹, |
| Film | in film | (μm) | 37° C.) |
| Inventive film 1 | EVOH | 104 | <1 |
| Inventive film 2 | EVOH | 73 | <1 |
| Inventive film 3 | EVOH | 86 | <1 |
| Control film 1 | pVDC | 75 | 17 |
| Control film 2 | pVDC | 63 | 17 |
| Control film 3 | pVDC | 75 | 17.5 |
| Control film 4 | Polyamide | 289 | 464 |

The results demonstrated that multilayer barrier films of the invention display superior hydrogen sulphide odour impermeability compared to control films. In all cases, inventive films demonstrated at least a 17-fold reduction in hydrogen sulphide permeation compared to control films; and in comparison to control film 4, films of the invention displayed an at least 464-fold reduction in hydrogen sulphide permeation.

The results confirm the excellent odour barrier properties of films of the invention, which make them especially suited to ostomy applications.

The above embodiments are described by way of example only. Many variations are possible without departing from the scope of the invention as defined in the appended claims.

1. An ostomy pouch comprising a cavity for containing human waste defined by a multilayer barrier film comprising in order from adjacent to the cavity outwards: an inner polymer carrier layer; an inner tie layer; a barrier layer comprising poly(ethylene-co-vinyl alcohol); an outer tie layer; and an outer polymer carrier layer; wherein the tie layers each comprise recycled polymer material and at least one compatibilizer.

2. An ostomy pouch as claimed in claim 1, wherein at least one polymer carrier layer comprises at least one polymer that is independently chosen from: a polyolefin, a polyamide, and combinations thereof, and wherein at least one polymer carrier layer preferably comprises at least one polymer that is independently chosen from: polyethylene, an ethylene copolymer, polypropylene, a propylene copolymer, and combinations thereof.

3. An ostomy pouch as claimed in claim 2, wherein at least one polymer carrier layer, and preferably the inner polymer carrier layer comprises polyethylene, preferably metallocene-catalysed polyethylene, and more preferably metallocene-catalysed linear low-density polyethylene.

4. An ostomy pouch as claimed in claim 2 or 3, wherein at least one polymer carrier layer, and preferably the outer polymer carrier layer comprises at least one copolymer of ethylene with at least one of: hexene, octene, vinyl acetate, and combinations thereof, wherein the ethylene copolymer is preferably ethylene-vinyl acetate.

5. An ostomy pouch as claimed in any preceding claim, wherein the inner polymer carrier layer comprises a polymer material that is more impermeable to moisture than the polymer material of the outer carrier layer.

6. An ostomy pouch as claimed in any preceding claim, wherein at least one polymer carrier layer has a thickness of between 4-18 microns.

7. An ostomy pouch as claimed in any preceding claim, wherein at least one tie layer comprises the at least one compatibilizer in a total amount of between 5-30 wt. % of the tie layer, preferably between 20-30 wt. % of the tie layer.

8. An ostomy pouch as claimed in any preceding claim, wherein at least one tie layer comprises the recycled polymer material in a total amount of between 50-100 wt. %, or preferably between 70-80 wt. %.

9. An ostomy pouch as claimed in any preceding claim, wherein the at least one compatibilizer comprises a maleic anhydride copolymer, preferably a copolymer of maleic anhydride with ethylene, and more preferably poly(ethylene-alt-maleic anhydride).

10. An ostomy pouch as claimed in any preceding claim, wherein the recycled polymer material comprises poly(ethylene-co-vinyl alcohol).

11. An ostomy pouch as claimed in any preceding claim, wherein the recycled polymer material comprises at least one polymer that is independently chosen from: a polyolefin, a polyamide, and combinations thereof.

12. An ostomy pouch as claimed in any preceding claim, wherein the recycled polymer material comprises polymer film material, preferably multilayer polymer film material.

13. An ostomy pouch as claimed in claim 12, wherein the recycled polymer material comprises material from a multilayer polymer film comprising a poly(ethylene-co-vinyl alcohol) barrier layer.

14. An ostomy pouch as claimed in claim 12 or 13, wherein the recycled polymer material comprises material from a multilayer polymer film comprising a tie layer comprising recycled polymer material and at least one compatibilizer.

15. An ostomy pouch as claimed in claim 14, wherein the recycled polymer material comprises material from a multilayer polymer film comprising: at least one polymer carrier layer; at least one tie layer comprising recycled polymer material and at least one compatibilizer; and at least one barrier layer comprising poly(ethylene-co-vinyl alcohol).

16. An ostomy pouch as claimed in any one of claims 12 to 15, wherein the recycled polymer material comprises material from a multilayer polymer film comprising a polymer carrier layer comprising at least one polymer that is independently chosen from: a polyolefin, a polyamide, and combinations thereof.

17. An ostomy pouch as claimed in any one of claims 12 to 16, wherein the recycled polymer material comprises unused polymer film material.

18. An ostomy pouch as claimed in any one of claims 12 to 17, wherein the recycled polymer material comprises polymer film edge trim.

19. An ostomy pouch as claimed in any one of claims 12 to 18, wherein the recycled polymer material comprises ostomy film material.

20. An ostomy pouch as claimed in claim 19, wherein the recycled polymer material comprises ostomy pouch film material, preferably unused ostomy pouch film material.

21. An ostomy pouch as claimed in any preceding claim, wherein at least one tie layer has a thickness of between 4-10 microns.

22. An ostomy pouch as claimed in any preceding claim, wherein the poly (ethylene-co-vinyl alcohol) of the barrier layer comprises between 10-50% vinyl alcohol units.

23. An ostomy pouch as claimed in any preceding claim, wherein the barrier layer has a thickness of between 3-15 microns.

24. An ostomy pouch as claimed in any preceding claim, wherein the multilayer barrier film further comprises: an outermost weld layer positioned furthest from the pouch cavity; and/or an innermost weld layer positioned nearest the pouch cavity.

25. An ostomy pouch as claimed in any preceding claim, wherein the multilayer film is a cast film.

26. An ostomy pouch as claimed in any preceding claim, wherein the multilayer film has a thickness of between 40-130 microns.

27. An ostomy pouch as claimed in any preceding claim, wherein the ostomy pouch comprises a front wall and a rear wall, wherein the front and rear walls are sealed together to form the cavity of the pouch therebetween, and wherein the front and rear wall comprise the multilayer barrier film.

28. A method of manufacturing an ostomy pouch comprising the steps of:
a. Providing a front wall and a rear wall, wherein the front and rear walls comprise a multilayer barrier film comprising in order: a first polymer carrier layer; a first tie layer comprising recycled polymer material and at least one compatibilizer; a barrier layer comprising poly(ethylene-co-vinyl alcohol); a second tie layer comprising recycled polymer material and at least one compatibilizer; and a second polymer carrier layer; and
b. Sealing the front and rear walls together to form a cavity therebetween for containing human waste such that the second polymer carrier and tie layers are located further from the cavity than the first polymer carrier and tie layers;
wherein the method further comprises providing an inlet for receiving human waste in the rear wall before or after step (b).

29. A multilayer barrier film comprising in order: a first polymer carrier layer; a first tie layer; a barrier layer comprising poly(ethylene-co-vinyl alcohol); a second tie layer; and a second polymer carrier layer; wherein the tie layers each comprise recycled polymer material and at least one compatibilizer, the at least one compatibilizer being present in a total amount of between 5-30 wt. % of each tie layer.

30. A method of manufacturing a multilayer barrier film comprising the steps of:
a. Providing a first and second carrier layer polymer composition;
b. Providing a barrier layer polymer composition comprising poly (ethylene-co-vinyl alcohol);
c. Providing a first and second tie layer polymer composition by combining recycled polymer material and at least one compatibilizer, the at least one compatibilizer being present in a total amount of between 5-30 wt. % of each tie layer polymer composition; and
d. Co-extruding the first and second carrier layer, barrier layer, and first and second tie layer polymer compositions to provide the multilayer barrier film, such that the film comprises in order: the first polymer carrier layer; the first tie layer; the barrier layer; the second tie layer; and the second polymer carrier layer.
